(19) United States
(12) Patent Application Publication
(10) Pub. No.: US 2021/0338953 A1
(43) Pub. Date: Nov. 4, 2021

(54) System and Method For Ventilating a Person

(71) Applicant: BreathDirect, Inc., Long Beach, CA (US)

(72) Inventors: Adam Marten, Long Beach, CA (US); Darren Saravis, Long Beach, CA (US)

(21) Appl. No.: 17/306,892

(22) Filed: May 3, 2021

Related U.S. Application Data
(60) Provisional application No. 63/019,325, filed on May 2, 2020.

(51) Int. Cl. A61M 16/00; A61M 16/20

(52) U.S. Cl. CPC A61M 16/024; A61M 16/208; A61M 2202/0208; A61M 2016/0027; A61M 2016/003; A61M 16/0072

(57) ABSTRACT

A ventilator is provided that dynamical adjusts the pressure, flow, and volume of the delivered gas to a patient as the condition of the patient changes. The ventilator adjusts the flow and mixing of gases through flow control valves. The ventilator includes at least two banks of valves, each bank having a plurality of valves, where each valve in the bank has a specific orifice size that is different from at least one other valve in the respective bank of valves. In one example, the ventilator further includes an exhalation valve assembly having an exhalation valve housing and exhalation valve base coupled together to retain a flexible exhalation tube. The exhalation valve assembly including at least two pistons extending at least partially through the exhalation valve assembly to contact the exhalation tube and, when actuated, impart pressure on the walls of flexible exhalation tube to restrict or completely close the flow of air through the flexible exhalation tube.

### CROSS REFERENCE TO RELATED APPLICATIONS

This application claims priority to U.S. Provisional Patent Application Ser. No. 63/019,325 filed May 2, 2020, titled System and Method for Ventilating a Person, which application is incorporated herein in its entirety by reference.

### FIELD OF THE INVENTION

The present invention is related in general to methods and systems for ventilating patients, and more particularly to an improved design for a medical ventilator and an improved method for controlling a ventilator.

### BACKGROUND OF THE INVENTION

Medical ventilators provide artificial respiration to patients whose breathing ability is impaired. Ventilators generally deliver breath to a patient from a pressured gas source. The use of ventilator is considered life-sustaining/life-supporting.

Known ventilators typically include a pneumatic system that delivers and extracts gas pressure, flow and volume characteristics to the patient and a control system (typically consisting of knobs, dials and switches) that provides an interface to a treating clinician. Optimal support of the patient's breathing requires adjustment by the clinician of the pressure, flow, and volume of the delivered gas as the condition of the patient changes. Such adjustments, although highly desirable, are difficult to implement with known ventilators as the ventilator demands continuous attention and interaction from the clinician.

The Ventilator Emergency Use Authorization (EUA) was issued in response to concerns relating to insufficient supply and availability of FDA-cleared ventilators for use in healthcare settings to treat patients during the Coronavirus Disease 2019 (COVID-19) pandemic.

According, a need exists for a ventilator that allow for ease of manufacturability to allow for rapid scale up of large volume production numbers. By allowing for large scale manufacturing, devices will be available to fulfil urgent needs for the product. Increased production volumes of the same type of ventilator will allow user facilities to acquire ventilators of the same type to fill any shortages, which will reduce user training needs and minimize the need to operate numerous types of ventilators.

### SUMMARY

An electronically controlled pneumatic ventilation system is provided that dynamical adjusts the pressure, flow, and volume of the delivered gas to a patient as the condition of the patient changes. The ventilator adjusts the flow and mixing of gases through flow control valves. The ventilator includes at least two banks of valves, each bank has a plurality of valves, where each valve in the bank has a specific orifice size that is different from at least one other valve in the respective bank of valves. In one example, the ventilator further includes an exhalation valve assembly having an exhalation valve housing and exhalation valve base coupled together to retain a flexible exhalation tube. The exhalation valve assembly including at least two pistons extending at least partially through the exhalation valve assembly to contact the exhalation tube and, when actuated, to impart pressure on the walls of flexible exhalation tube to restrict or completely close the flow of air through the flexible exhalation tube.

In one example of an implementation, a ventilator is provided to dynamical adjustment the pressure, flow, and volume of the delivered gas to a patient as the condition of the patient changes. The comprising a ventilator console; and an exhalation valve housing and exhalation valve base for coupling with the exhalation valve housing to retain a flexible exhalation tube and at least two pistons extending through the exhalation valve base for imparting pressure on the flexible exhalation tube when actuated to restrict the flow of air through the flexible exhalation tube. The ventilator may further include an anvil coupled to each of the at least two pistons that move with the piston to impart pressure on the walls of the flexible tube and to push the walls of the flexible exhalation tube toward one another and at least partially or fully restricting the flow of air through the flexible exhalation tube by partially or fully closing the tube. The piston and anvil can be replaced with another mechanism for restricting the flow of air through the tube at least partially or fully, without departing from the scope of the invention. Devices capable of squeezing the tube or moving one or both walls toward one another to restrict or minimize the air flow through the tube are within the scope of the invention.

In another example, the ventilator able to dynamical adjustment the pressure, flow, and volume of the delivered gas to a patient as the condition of the patient changes comprises: a ventilator console; an exhalation valve assembly; and flow control valves for controlling the flow and mixing the gas. The flow control valves include at least two banks of valves, each bank have a plurality of valves where each valve has a specific orifice size that is different from at least one other valve in the respective bank of valves. In this example, the valves may be solenoid valves, and at least one of the banks of valves delivers oxygen and at least one of the banks of valves may deliver air. In other examples, each of the plurality of valves in at least one of the at least two banks of valves has a different orifice size. In operation, the plurality of valves provides a total flow control up of at least 101 LPM.

In yet another example, flow control valves for controlling the flow and mixing the gas in the ventilator has at least two banks of valves and each bank of valves has at least seven (7) solenoid valves that each provide controlled flow from 0-37 LPM per valve, such that each valve in the bank has a specific orifice size to provide necessary flow with an upstream regulated pressure of at least 10 PSIg and a total flow control of at least 101 LPM.

Other devices, apparatus, systems, methods, features and advantages of the disclosure will be or will become apparent to one with skill in the art upon examination of the following figures and detailed description. It is intended that all such additional systems, methods, features and advantages be included within this description, and be protected by the accompanying claims.

### DESCRIPTION OF THE FIGURES

The invention may be better understood by referring to the following figures. The components in the figures are not necessarily to scale, emphasis instead being placed upon illustrating the principles of the invention. In the figures, like reference numerals designate corresponding parts throughout the different views.

FIG. 1 is front perspective view of a ventilator system of the present invention.

FIG. 2 is an example of a display screen on the user interface of the ventilator system.

FIG. 3 is a top perspective view of the exhalation valve assembly of the ventilator system of FIG. 1 attached to the ventilator console.

FIG. 4 is an exploded view of the exhalation valve assembly of FIG. 3.

FIG. 5 is an exploded view of the exhalation valve assembly of FIG. 3 showing the placement of the exhalation value in exhalation valve housing.

FIG. 6 is an exploded view of the exhalation valve assembly of FIG. 3 with the exhalation value in the exhalation valve housing.

FIG. 7 is a top perspective view of the exhalation valve assembly of the ventilator system of FIG. 1 detached from the ventilator console.

FIG. 8a is a top perspective view of the cross-section of the exhalation valve assembly of the ventilator system of FIG. 1 taken along line 8-8 of FIG. 7, which is a horizontal cross section with the top half removed, showing both pistons open or both pistons in the resting position.

FIG. 8b is a top perspective view of the cross-section of the exhalation valve assembly of the ventilator system of FIG. 1 taken along line 8-8 of FIG. 7, with at least one piston actuated.

FIG. 8c is a top perspective view of the cross-section of the exhalation valve assembly of the ventilator system of FIG. 1 taken along line 8-8 of FIG. 7 shown both pistons activated.

FIG. 9 is rear perspective view of a ventilator system of the present invention.

FIG. 10 is a block diagram showing the various components of the ventilator system of the present invention.

FIG. 11 is a schematic of example ventilator controls for the delivery of the mixed gas to the patient.

### DETAILED DESCRIPTION

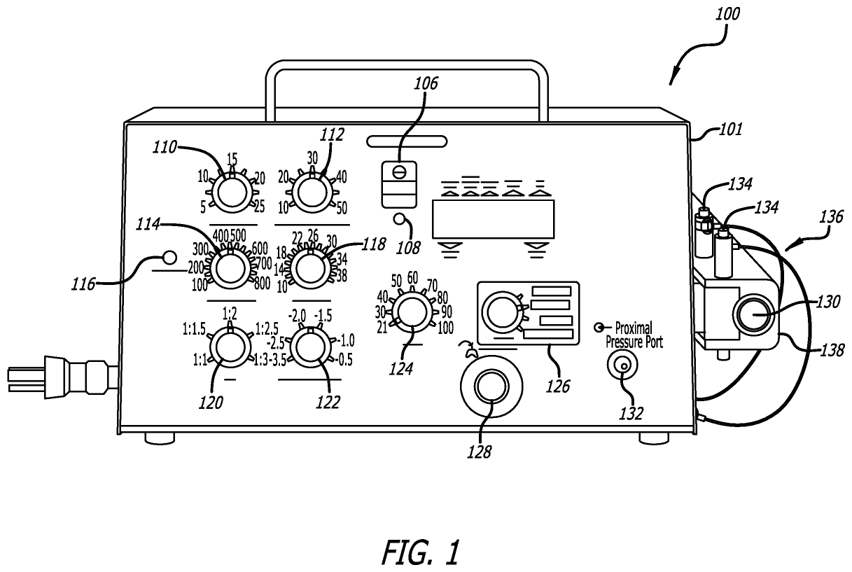

As illustrated below and in the attached FIGS. 1-11, the present invention relates to an electronically controlled pneumatic ventilation system 100. In describing the ventilation system 100 of the present invention and its operation, several common acronyms may be used, including but not limited to the following: (1) PC—Pressure Control; (2) VC—Volume Control; (3) FiO2—Fraction of Inspired Oxygen; (4) I:E ratio—Inspiration to Expiration Ratio; (5) RR—Respiratory Rate, (6) PEEP—Positive End-Expiatory Pressure, (7) Pplateau—Plateau Pressure; (8) PIP—Peak Inspiratory Pressure; and (9) Tv—Tidal volume.

The ventilation system or ventilator 100 of the present invention is comprised of a ventilator console 101 having a user interface (UI) 102 and, as described in more detail below, electronic circuits with software to control various valves and use feedback gathered through sensors to control the delivery of ventilation therapy. The ventilator 100 may include Bluetooth or other networking connectivity features, but such features are not required, especially for rapid production required in response to pandemic conditions. The ventilator 100 may be powered by AC power and contain a battery backup to protect against power failure or unstable power and to facilitate brief intrahospital transport. The ventilator 100 is design to automatically switch to battery power if AC power is lost. This switch will trigger a “loss of main power” alarm. The battery shall automatically recharge when the ventilator 100 is connected to suitable line power.

FIG. 1 illustrates a front perspective view of one example of a ventilator 100 including a user interface 102 that contains turn knobs for controlling the ventilator settings and an integrated display screen 104 for pressure monitoring and alarm notification 106 (which may include an alarm audio silenced button). The ventilator 100 is operated by user input through control knobs.

A breath indicator light 108 illuminates when a ventilation/breath is being delivered. The ventilator 100 is operated by user input through control knobs. These inputs control the pneumatics to deliver a controlled gas mixture to the patient. The ventilator 100 contains sensors to monitor flow and pressure and adjust delivery to the patient according to the user-selected setting. Monitored data is displayed on the graphical user interface display screen 108.

As illustrated, the ventilator 100 may include a PEEP control knob 110, and inspiration pressure control knob 112, a tidal volume control knob 114, set tidal volume alarm button 116, rate control knob 118, I:E control knob 120, sensitivity control knob 122, 02 control knob 124, ventilation mode control knob 126, inspiratory port (to patient) 128, expiration port (from patient) 130, pressure sensor connection 132, assembly pins 134 and exhalation valve assembly 136. Through the display screen 104, various control knobs, and switches, the user can set the ventilation therapy mode and read any applicable data from the ventilator display screen 104, which may be a low-resolution display screen 104. The display screen 104 conveys the patient-monitored parameters, as well as any alarms, including the priority of the alarm, and whether the alarm is paused.

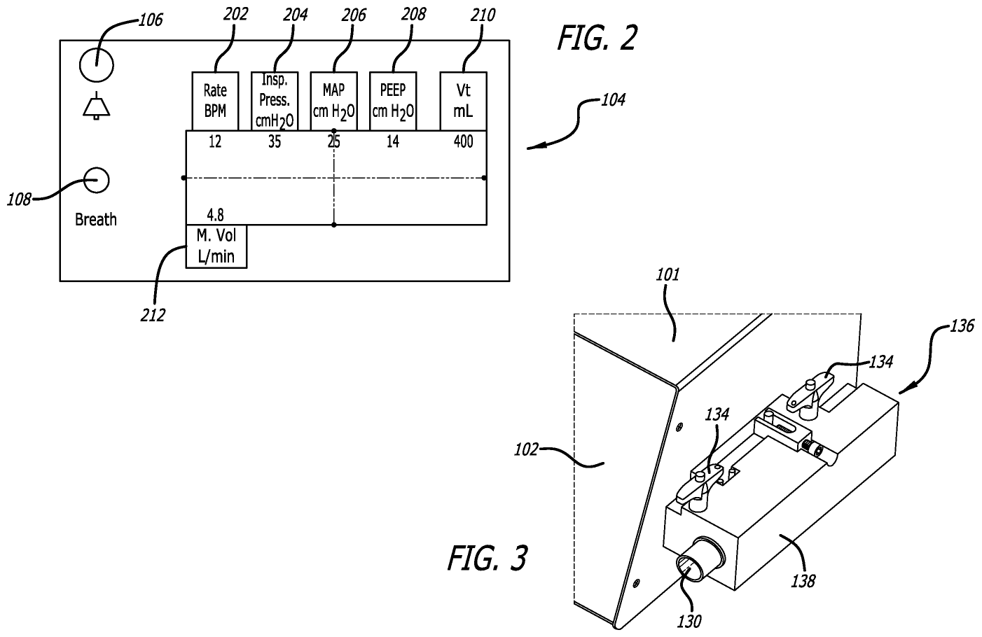

FIG. 2 illustrates one an example of a display screen 104 on the user interface 102 of the ventilator console 101 of a ventilator system 100. In the illustrated example, a ventilator display screen 104, alarm indicator with audio silenced button 106 and a breath indicator light 108 is illustrated.

As illustrated, the display screen 104 may display the following: (1) Rate (Respiratory Rate) [breaths per minute (BPM)] 202; (2) Insp. Press. (Inspiratory Pressure) [cmH₂O] 204; (3) MAP (Mean Airway Pressure) [cmH₂O] 206; (4) PEEP (Positive End-Expiratory Pressure) [cmH₂O] 208; (5) Vt (Tidal Volume) [mL] 210; (6) M. Vol. (Minute Volume) [L/min] 212. All gas volume, flow and leakage specifications are expressed at standard temperature and pressure (STDP) except those associated with the Ventilator Breathing System (VBS) are expressed at body temperature and pressure (BTPS).

Although not shown, the display screen 104 may display messages. The display screen 104 may provide alarm displays, with alarm priority marks, an alarm audio paused display and alarm instructions. In one example implementation, the message “NO VENTILATION” may be displayed when the ventilation mode is set to Standby. The message “AUDIO PAUSED” may be displayed if the alarm audio paused button is pressed to temporarily silence an alarm. A ventilator 100 may be equipped with any one or more of the following alarms: (1) device power alarm; (2) Disconnect Alarm (Low peak inspiratory pressure (PIP) alarm); (3) External power supply failure Alarm; (4) Back-up Battery; (5) Low Fraction of Inspired Oxygen (FiO2) alarm; (6) Maximum peak inspiratory pressure (PIP) alarm; (7) Occlusion alarm/Continuing Pressure; (8) tidal volume (Tv) not met/exceeded alarm; (9) optionally, hypoventilation; (10) positive end-expiatory pressure (PEEP) alarm; (11) leakage alarm; and (12) optionally, a carbon dioxide monitor must be used for the measurement of expired CO₂. A Low FiO2 alarm may not be included if the ventilator does not include an oxygen monitor; however, a third party oxygen monitor should still be used with the device. All data applicable to the operation of the ventilator 100, including the ventilator settings, ventilator modes, sensor readings, battery levels, alarm modes, and power present may be displayed on the display screen 104.

The ventilator 100 may also include audible and visual alarms indicating abnormal conditions or technical issues with the ventilator 100. The ventilator 100 can perform a self-test upon startup to check the ventilator 100 and check all alarm features, to confirm proper functioning of the ventilator 100. If a condition is critical enough to possibly compromise the safe ventilation of the patient, the ventilator 100 may be placed into an ambient state that disables power to the valves, allowing the patient to inspire and exhale room air through the exhalation valve.

The ventilator of the present invention has three ventilation modes: (i) Pressure Regulated Volume Control (PRVC); (ii) Pressure Controlled-Continuous Mandatory Ventilation (PC-CMV); and (iii) Bilevel Positive Airway Pressure-Spontaneous/Timed (BPAP-S/T). The mode select button 126 allows a user to switch operation of the ventilator system 100 between these three modes, as well as a standby mode. The mode selection is controlled by the user with the mode selection switch 126.

PRVC (Pressure regulated volume control) mode is where the clinician sets a desired tidal volume and the output is a pressure regulated flow to achieve this volume. If, at the end of the breath the volume is less than or greater than the desired, the pressure is adjusted for the next breath. PRVC delivers a set tidal volume at a set respiratory rate, but also responds to a patient's inspiration. A spontaneous inspiratory effort triggers a full-tidal-volume breath and resets the timer for the subsequent mechanical breath.

Either the ventilator 100 or the patient can initiate breaths, and a constant pressure, like that of pressure-control ventilation (PCV), is applied throughout both mechanical and patient breaths. The ventilator 100 monitors each breath, comparing the delivered tidal volume with the set tidal volume and then adjusting inspiratory pressure to achieve delivery of the set tidal volume. If the delivered tidal volume is too low, the inspiratory pressure of the next breath is increased. If it is too high, the pressure of the subsequent breath is decreased.

PC-CMV mode is a mandatory ventilation mode that delivers a set constant pressure at a constant rate to the patient. The patient cannot initiate breaths. Tidal Volume is controlled by the pressure settings between PIP and PEEP. Tidal Volume alarm limits and pressure alarm limits provide notification of changes in patient ventilation. The tidal volume is dependent on the pressure difference between PEEP and PIP, the lung mechanics, and patient breathing effort. The number of mandatory breaths is time cycled and is not triggered by the patient. The patient can breathe in between the mandatory breaths or at any point during the breath cycle, but the breaths are not assisted by the ventilator.

BPAP-S/T mode is a bilevel pressure support mode that supports spontaneous breathing of the patient. If the patient does not initiate a breath within an apneic time window, the ventilator 100 delivers a mandatory breath, and resets the apneic time window. BPAP-S/T is an invasive or non-invasive ventilation mode that provides two levels of inspiratory positive pressure. BPAP-S/T ventilation mode defaults to Spontaneous (S) mode, so when the patient initiates inhalation, the device triggers IPAP (Inspiratory Airway Pressure), which is set by the Insp. Pressure control knob 112. Then when the patient exhales, the device changes to EPAP (Expiratory Positive Airway Pressure), which is set by the PEEP control knob 110. The instrument supports patient breathing.

The BPAP-S/T ventilation mode is intended to be used to provide ventilation support to patients who may benefit from positive pressure, invasive or non-invasively. This mode provides ventilation support and alarm notification for patients who stop spontaneously breathing. In BPAP-S/T ventilation mode, the system will default to Tidal Volume alarm limits of ±50% of the current setpoint. Tidal Volume upper and lower alarm limits can be user adjusted. PC-CMV (Pressure Controlled-Continuous Mandatory Ventilation). The Timed (T) mode automatically initiates a backup rate if the patient does not breathe within a 15 second apneic window, then the instrument will breathe at the minimum number of breaths per minute. The instrument will post an alarm (low or medium alarm, non-latching) that it is in mandatory ventilation. If the patient starts to breathe on their own, again the instrument will detect this and go back to supporting spontaneous breath and the alarm would self-clear.

Example ventilator operational parameter ranges are found below.

| Parameter | Range |
|---|---|
| Respiratory Rate (RR) | 10-30 |
| (BPM) | |
| Tidal Volume (T_{V}) | 250-800 |
| (mL) | |
| Flow Rate (L/min) | 100 |
| Insp. Pressure (cm | 10-50 |
| H₂O) | |
| positive end-expiatory | 5-25 |
| pressure (PEEP) (cm | |
| H₂O) | |
| Available Ventilation | PRVC, CMV-PC, |
| Modes | BPAP-S/T |
| Air source | Hospital Air |

Below is a table that provides example ventilator settings used for different ventilator modes.

| | Ventilation Mode | | | |
|---|---|---|---|---|
| Setting | Knob Settings | PRVC | BPAP-S/T | PC-CMV |
| LED Color | — | Blue | Amber | Green |
| PEEP | 5 to 25 cmH₂O; | Active | Active (EPAP) | Active |
| | 2.5 cmH₂O increments | | | |
| Insp. Pressure | 10 to 50 cmH₂O; | Active; User-set | Active (IPAP) | Active |
| | 5 cmH₂O increments | upper limit | | |
| Tidal volume | 250 to 800 mL; | Active | Passive; User set | Passive; User set |
| | 50 mL increments | | upper and lower | upper and lower |
| | | | alarm limits | alarm limits |
| Rate | 10 to 30 BPM; | Active | Active | Active |
| | 2 BPM increments | | | |
| I:E | 1:1 to 1:3; | Active | Active | Active |
| | 1:0.5 increments | | | |
| Sensitivity | −3.0 to −0.5 cmH₂O; | Active | Active | Inactive |
| | 0.5 cmH₂O increments | | | |
| Oxygen | 21, 30, 40, 50, 60, 70, | Active | Active | Active |
| | 80, 90, 100% | | | |

The Positive end-expiratory pressure (PEEP) is indicated in cmH₂O. To adjust the PEEP setting Set PEEP value by turning the control knob 110 on the front panel of the ventilator console 101 to the desired value. Monitor the PEEP readout on the monitor for several breath cycles to be certain it is maintained. If PEEP is low, turn PEEP control knob 110 slightly to the right to create more backpressure. Monitor the PEEP readout on the monitor for several breath cycles to be certain it is maintained. If PEEP is low, turn PEEP control knob 110 slightly to the right to create more backpressure. Monitor the PEEP readout on the monitor for several breath cycles to be certain it is maintained. If PEEP is high, turn the PEEP control knob 110 slightly to the left to open the exhalation valve and reduce resistance. Monitor the PEEP readout on the monitor for several breath cycles to be certain it is maintained. For BPAP-S/T ventilation mode, this dial sets expiratory positive airway pressure (EPAP).

Inspiratory pressure (Insp. Pressure) is indicated in cmH₂O. To set the Insp. Pressure, Set Insp. Pressure value by turning the control knob 112 on the front panel of the ventilator console to the desired value. Monitor the Insp. Press. readout on the monitor for several breath cycles to be certain it is maintained. If Insp. Press. is low, turn the Insp. Pressure control knob 112 slightly to the right to create more inspiratory pressure. Monitor the Insp. Press. readout on the monitor for several breath cycles to be certain it is maintained. If Insp. Press. is high, turn the Insp. Pressure control knob 112 slightly to the left to reduce the inspiratory pressure. Monitor the Insp. Press. readout on the monitor for several breath cycles to be certain it is maintained. For BPAP-S/T ventilation mode, the Insp. Pressure dial 112 sets inspiratory positive airway pressure (IPAP). For PRVC ventilation mode, the Insp. Pressure dial 112 sets the inspiratory pressure upper limit. Tidal volume delivered during inspiration is indicated in milliliters (mL).

To set the tidal volume, turn the control knob 114 on the front panel of the ventilator console to the desired value. Monitor the tidal volume readout on the monitor for several breath cycles to be certain it is maintained. If tidal volume is low, turn the tidal volume control knob 114 slightly to the right to increase tidal volume. Monitor the tidal volume readout on the monitor for several breath cycles to be certain it is maintained. If tidal volume is high, turn the tidal volume control knob 114 slightly to the left to reduce tidal volume. Monitor the tidal volume readout on the monitor for several breath cycles to be certain it is maintained. Tidal volume is not an active input for BPAP-S/T or PC-CMV mode. In these modes, the tidal volume control knob 114 is used to set the upper and lower tidal volume alarm limits.

To set the tidal volume alarm limits (BPAP-S/T or PC-CMV ventilation modes only), press the “Set Vt Limit” button next 116 to the tidal volume control knob 114. Interact with the display screen 104 to verify what limit you are setting (e.g. upper, lower). Move the tidal volume control knob 114 to set the upper alarm limit. Press the “Set Vt Limit” button next to the tidal volume control knob 114 to confirm the upper tidal volume alarm limit that is set. Press the “Set Vt Limit” button 116 next to the tidal volume control knob 114 to switch to the lower tidal volume alarm limit. Move the tidal volume control knob 114 to set the lower alarm limit. Press the “Set Vt Limit” button 116 next to the tidal volume control knob 114 to confirm the lower tidal volume alarm limit is set. Press the “Set Vt Limit” button 116 next to the tidal volume control knob 114 to exit out of the alarm setting mode and show the alarm values set. Verify the alarm limits are set as desired. The alarm settings will revert to the previous alarm settings for tidal volume if no entry is made or there is no change in values for 10 seconds. The tidal volume alarm limits may be set to default to 50% above and below the current tidal volume knob location for PC-CMV and BPAP-S/T on power up unless otherwise specified by the user.

To view previously set or default tidal volume alarm limits (BPAP-S/T or PC-CMV ventilation modes only), a user may quickly press the “Set Vt Limit” button 116 next to the tidal volume control knob 114 two times. Check the display screen for the current upper and lower limit settings. This display will automatically clear and return to the previous display after 3 seconds. If you wish to change the lower and upper alarm limit settings, press the “Set Vt Limit” button 116 next to the tidal volume control knob 114 once and follow instructions above for setting the tidal volume alarm limits.

Breathing rate is indicated in breaths per minute (BPM). To set the value of rate, turn the dial 118 to desired value prior to beginning ventilations. Set rate value by turning the control knob 118 on the front panel of the ventilator console to the desired value. Monitor the rate readout on the monitor for several breath cycles to be certain it is maintained. If rate is low, turn the rate control knob 118 slightly to the right to increase breathing rate. Monitor the rate readout on the monitor for several breath cycles to be certain it is maintained. If rate is high, turn the rate control knob 118 slightly to the left to reduce breathing rate. Monitor the rate readout on the monitor for several breath cycles to be certain it is maintained.

Inhalation-exhalation ratio (I:E) is expressed in a numeric ratio (e.g. 1:2). The control knob 120 can achieve an I:E ratio of 1:1 to 1:3. To set the value of the I:E ratio, turn the dial 120 to the desired values prior to beginning ventilations.

Sensitivity sets the trigger pressure for initiating patient breathing support, and is indicated in cmH₂O. It should be set by a respiratory therapist, pulmonologist, or other physician. To set the value of the sensitivity, turn the control knob 122 to the sensitivity value when setting other parameters. Sensitivity is not adjusted in PC-CMV mode.

Oxygen control knob 124 is given in percentages (%) and is able to be adjusted, for example, at increments of 10%. For example, at 02=100%, no air is provided to the patient through the ventilator, only oxygen.

The ventilator interface 102 will monitor patient parameters on the display screen 104. These values are determined based on built-in sensors within the ventilator console 101. Pressure settings and readings are indicated on the ventilator system in cmH₂O. The Inspiratory Pressure (Insp. Press.) reading on the ventilator display shows the highest amount of pressure applied to the patient's chest and the circuit when the patient's lungs are filled with air.

In PRVC Mode, the Inspiratory Pressure reading 204 displays the patient Plateau Pressure for the ventilation. The Inspiratory Pressure reading 204 is indicated on the ventilator system in cmH₂O. The Positive-End Expiratory Pressure (PEEP) reading 208 on the ventilator display 104 shows the pressure remaining in the airways after the patient exhales (at the end of the respiratory cycle). The value provided is the difference between the measured pressure in the airways and the atmospheric pressure in mechanically ventilated patients. The PEEP reading 208 is indicated on the ventilator system in cmH₂O. The Mean Airway Pressure (MAP) reading 206 on the ventilator display 104 shows the average (mean) pressure the patient's lungs are exposed to during the entire respiratory cycle (both inhalation and exhalation). The MAP reading 206 is indicated on the ventilator system 100 in cmH₂O. The Tidal Volume (Vt) reading 210 on the ventilator display 104 shows the volume of gas delivered to the patient's lungs per ventilation. The Tidal Volume reading 210 is indicated on the ventilator system in milliliters (mL). The Minute Volume 212 reading on the ventilator display 104 shows the volume of gas delivered to the patient's lungs per minute. The Minute Volume reading 212 is indicated on the ventilator system in liters per minute (L/min). The Respiratory Rate (Rate) reading 202 on the ventilator display shows the number of breaths that are delivered per minute. The Rate reading is indicated on the ventilator system in breaths per minute (BPM).

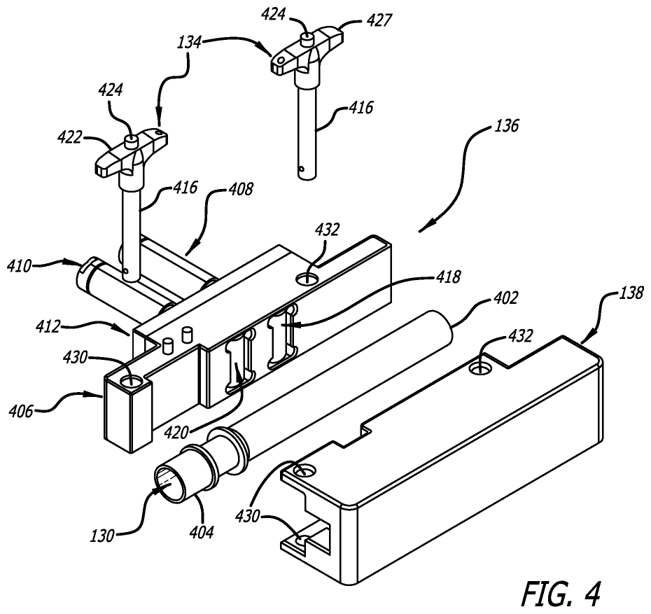

FIG. 3 is a top perspective view of the exhalation valve assembly 136 of the ventilator system 100 of FIG. 1 attached to the ventilator console. 101. The exhalation valve assembly 136 includes the exhalation valve 136, pins 134, and exhalation valve housing 138. FIG. 4 is an exploded view of the exhalation valve assembly 136 of FIG. 3 separated from the ventilator console 101.

The exhalation valve assembly 136 of the ventilation system 100 of the present invention includes exhalation valves or tubes 130 that are intended to be part of the disposable patient circuit. The exhalation valves 130 in the one example may include 22 mm inlet and outlet ports per ISO 5356, be disposable and have a resistance to flow that is no greater than 4.8 cmH₂O/L/s when the diaphragm is relaxed at a maximum flow of 60 L/m. Ideally, the exhalation valve noise level should not exceed 50 dB per ISO 80601-2-74:2017, 201.9.6.2.1.101, g. The exhalation valves 130 are made of biocompatible materials are intended to meet the requirements of ISO 18562 parts 1 through 4. The exhalation valves 130 mate directly with the conventional ventilator (no tubing). The exhalation valves 130 are packaged as a clean, non-sterile disposable. The exhalation valves' airway exit port is designed so no standard equipment can interface with the exit port. The exhalation valves 130 operate in any orientation when installed on the ventilator 100 of the present invention.

As illustrated in FIG. 4, the exhalation valve assembly 136 includes two pins 134, an exhalation valve housing 138, an exhalation valve 130, which includes a tube 402 and valve adapter 404, exhalation valve base 406, a first piston 408, second piston 410 and an exhalation valve mounting plate 412. The first and second pistons 408 and 410 extend through openings in the valve mounting plate 412 and exhalation valve base 406 to move a first anvil 418 and second anvil 420, respectively. The pins 134 include locking pins 416, T-handles 422 and buttons 424 for release the locking pins 416. The pins 134 extend though aligned holes 430 and 432 in the exhalation valve housing 136 and exhalation valve base 406 when coupled to maintain the exhalation valve 136 in the exhalation housing 136 during operation.

The pins 134 are used during operation to retain the exhalation valve 130 between the exhalation valve housing 138 and exhalation valve base 406. The first piston 408, during operation, is actuated to partially compress the exhalation tube 130, thereby limiting the airflow through the exhalation tube 130. The second piston 410 is actuated to completely compress the exhalation tube 130 during operation. The first piston 408 and second pistons 410 move first anvil 418 and second anvil for 20 forward when actuated to compress the exhalation valve 130. The exhalation valve housing 138 is a removable component of the exhalation valve 136 that a user may remove to replace the exhalation tube 130. The exhalation housing 138 provides support and retention of the exhalation tube 130 during operation. The exhalation valve housing 138 also provides safety protection from parts that are moving during operation.

The exhalation valve base 406 aligns with the exhalation valve housing 138 to mount the exhalation valve housing 138 to the exhalation valve base 406 to retain the exhalation valve tube 130 there between during operation. The exhalation valve base 406 also guides the first and second anvil 418 and 420 during operation and provides a stop for the first anvil 418. The exhalation valve mounting plate 412 provides a mounting location for the first and second piston 408 and 410. The exhalation valve mounting plate for 12 also provides a resting surface for the exhalation valve assembly 136 against the ventilator console 101 when mounted to the ventilator console 101.

FIG. 5 is an exploded view of the exhalation valve assembly of FIG. 3 showing the placement of the exhalation valve 130 in exhalation valve housing 138. FIG. 5 best illustrates how to insert the exhalation valve 130 in the exhalation valve assembly 136.

To install the exhalation valve 130, inspect the exhalation valve assembly 136 for debris or damage should first be performed. Any part with debris or damage should be replaced. The button 424 on the top of one pins 134 on the exhalation valve assembly 136 is pressed and simultaneously pull up on the pin 134 to remove. Repeat for the remaining pin 134 to remove. Pull back the exhalation valve housing. If an exhalation valve is currently in place, remove it and dispose of the used exhalation valve per your Institution's policies. Install a new single-use exhalation valve such that the end of the valve with the hard-plastic connector is oriented towards the front of the ventilator console. The groove closest to the front end of the plastic connector should be aligned so the front wall of the exhalation valve assembly housing fits within the groove. The outside ridge of the plastic connector should fit flush with the front of the exhalation valve housing.

In operation, to remove or install the exhalations valves 130, a user may press the button for 24 on the top of one of the pins on the exhalation valve assembly 136 and simultaneously pull up on the pin 134 to remove the pin. This is then repeated for the remaining pin 134 to remove the pin. A user can then pull back the exhalation valve housing 136, which may completely disengaged from the exhalation valve base 406, or be pivotally connected to the exhalation valve base 406 such that the exhalation valve housing 136 opens at one end away from the exhalation valve base 406. If an exhalation valve or tube 130 is currently in place, a user can then remove it and dispose of the used exhalation valve 130 and install a new single-use exhalation valve 130 such that the tube 404 with the hard-plastic connector or adapter 404 is oriented towards the front of the ventilator console 101. The groove closest to the front end of the plastic connector 404 should be aligned so the front wall of the exhalation valve assembly housing 138 fits within the groove. The outside ridge of the plastic connector 404 should fit flush with the front of the exhalation valve housing 138.

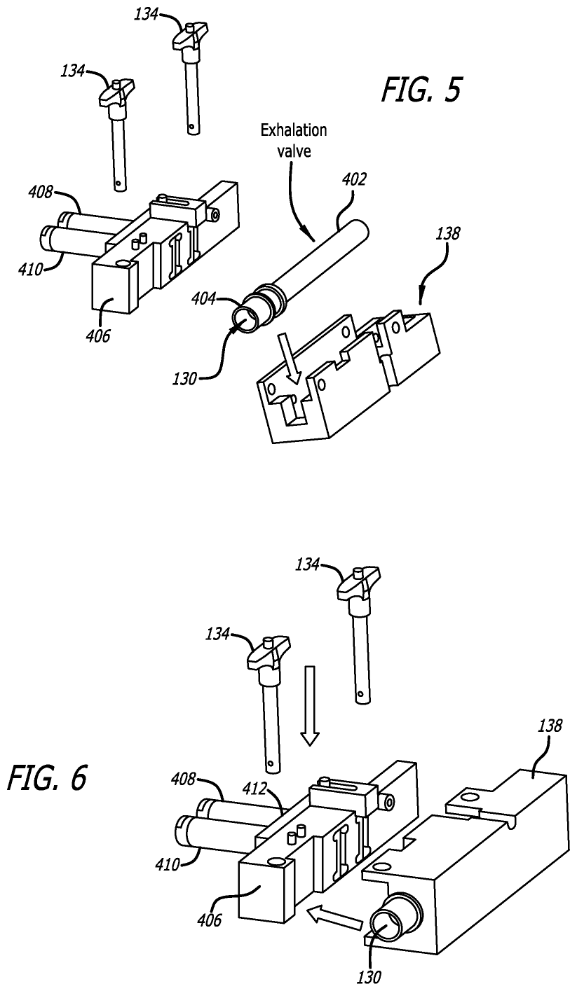

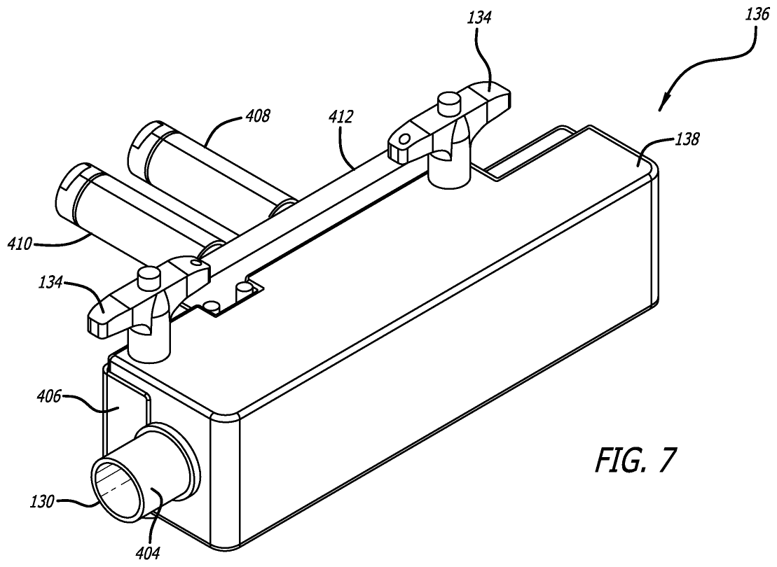

FIG. 6 is an exploded view of the exhalation valve assembly of FIG. 3 with the exhalation valve in the exhalation valve housing. FIG. 6 best illustrates how to assembly the exhalation valve assembly once the exhalation valve is inserted. To assemble the exhalation valve assembly once the valve 130 is in the exhalation valve housing 138, one closes the exhalation valve housing and secure by fully inserting the pins, pressing the top button, into the exhalation valve assembly. Release the top button and gently pull up on the handle of each pin to confirm pins are secured in place. Gently pull on the plastic exhalation tube connector to confirm that it is secured in place. If it is not secure, reopen the exhalation valve assembly and adjust the exhalation tube. FIG. 7 is a top perspective view of the exhalation valve assembly of the ventilator system of FIG. 1 shown fully assembled before attached to the ventilator console 101.

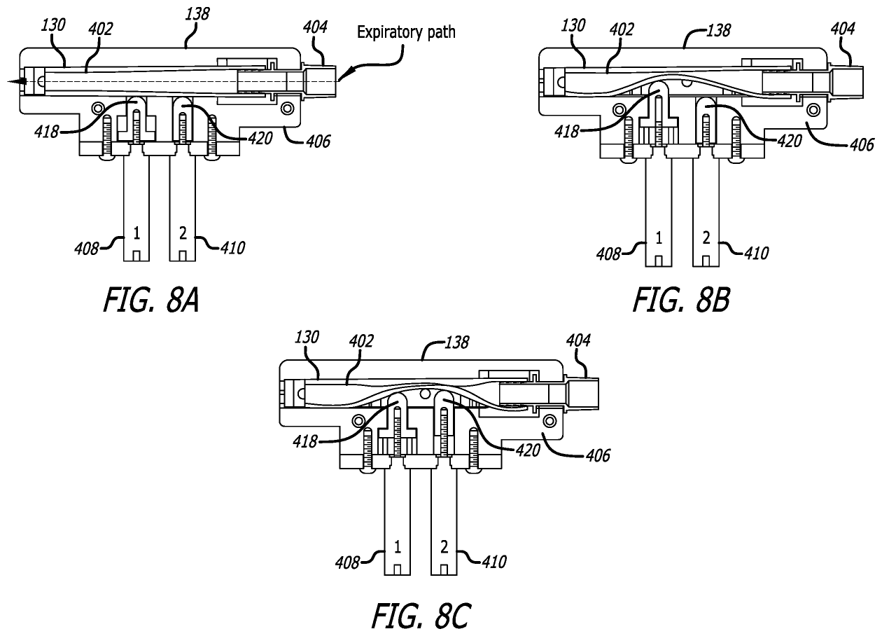

FIG. 8a is a top perspective view of the cross-section of the exhalation valve assembly 136 of the ventilator system 100 of FIG. 1 taken along line 8-8 of FIG. 7. FIG. 8a illustrates the first piston and second piston 408 and 410 and the first anvil and second anvil 418 and 420 in a resting and/or open position, allowing for the expiratory path to remain open. In this manner, when inhalation is required both pistons 408 and 410 will retract to allow full open flow of the expiratory path. Full flow is allowed when the pistons 408 and 410 are open or in the resting position. Pistons 408 and 410 are spring-loaded so that the pistons 408 and 410 retract to their open or resting position after engagement. Any loss of operational pressure to the ventilator 100 will return when the pistons 408 and 410 return to their resting position, which opens the exhalation airway completely and allows for the patient to inhale and exhale through the exhalation pathway.

FIG. 8b is a top perspective view of the cross-section of the exhalation valve assembly 136 of the ventilator system 100 of FIG. 1 taken along line 8-8 of FIG. 7. FIG. 8b illustrates the first piston 408 and first anvil 418 in an actuated position. This restricts flow through the exhalation tube 130. This position ensures proper PEEP is maintained. As illustrated in FIG. 8b, the first anvil 418 is only able to partially compress the tube 130, being restricted from fully entering the chamber holding the exhalation tube 130 by a forward wall in the exhalation valve base 406. Whereas, in contrast, as illustrated in FIG. 8c, which is a top perspective view of the cross-section of the exhalation valve assembly 136 of the ventilator system 100 of FIG. 1 taken along line 8-8 of FIG. 7, the second anvil 420 is able to extend fully into the chamber holding the exhalation valve 130 to completely close the expiratory pathway in order to achieve PIP, when the first piston 408 is also actuated. While the illustration shows the operation of the exhalation assembly 136 using two pistons 408 and 410, those skilled in the art will recognize that it is possible to use only one piston or more than two pistons without departing from the scope of the invention.

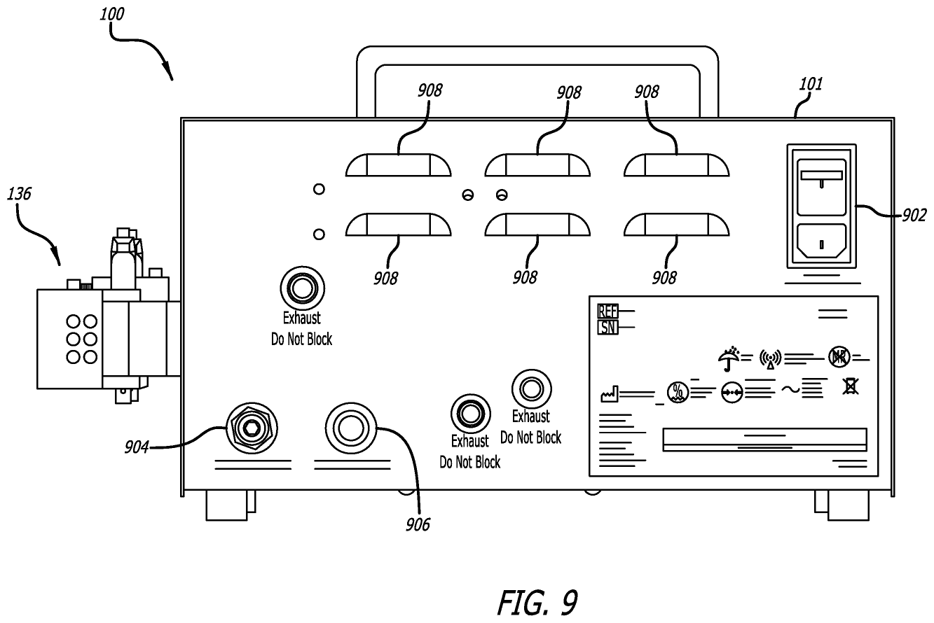

FIG. 9 is rear perspective view of a ventilator system of the present invention. The back of the ventilation console may include: (1) a power on/off switch 902; (2) an oxygen intake port 904; (3) an air supply intake port 906; (4) cooling vents 908; and (5) and AC power receptable 910. Both air and oxygen supply must be connected to the ventilator console 101 for operation of the system.

In this illustrated example, the oxygen intake port 904 and air supply inlet port 906 located on the back of the ventilator console 101 are standard Diameter Index Safety System (DISS) connection types. For operation, input pressure are optimally between 40-72 psi (276-496 kPa), with a maximum flow rate of 100 L/min. The air supply may be sourced from wall air or a medical air tank as long as the source air is output has a DISS air fitting or other fitting compatible with the ventilator console inputs, as well as a regulator to limit the input pressure to be between 40-72 psi (276-496 kPa).

To connect oxygen to the intake port, a standard DISS oxygen fitting and a regulator capable of limiting the input pressure to 40-72 PSI or 276-496 kPa is required. The oxygen supply may be sourced from the wall so long as the source features a standard Diameter Index Safety System (DISS) oxygen fitting. The oxygen may be alternatively supplied by an oxygen tank if it contains a standard Diameter Index Safety System (DISS) oxygen fitting and a regulator capable of limiting the input pressure to 40-72 PSI or 276-476 kPa. Input pressure must be between 40-72 PSI or 276-496 kPa at a maximum flow rate of 100 L/min.

During operation, the ventilator system 100 supplies gas to the patient via the inspiratory limb or port opening 128 on the front of the ventilator console (valve labeled “TO PATIENT”). The ventilator controls the oxygen ratio and the delivery of the mixed gas to the patient using electronic valves and feedback control through sensors, as further illustrated and discussed below.

The ventilator controls the oxygen ratio and the delivery of the mixed gas to the patient using electronic valves and feedback control through sensors. Oxygen (O2) is the percentage of oxygen concentration being delivered to the patient as a mixture of oxygen and/or air depending on the oxygen concentration percentage. As set forth above, the oxygen control knob is given in percentages (%) and is able to be adjusted, for example, at increments of 10%. For example, at O₂=100%, no air is provided to the patient through the ventilator, only oxygen. The control output is sent to a bank of blender valves, which use orifices to set the selected O₂ percentage as a ratio of oxygen and air gases.

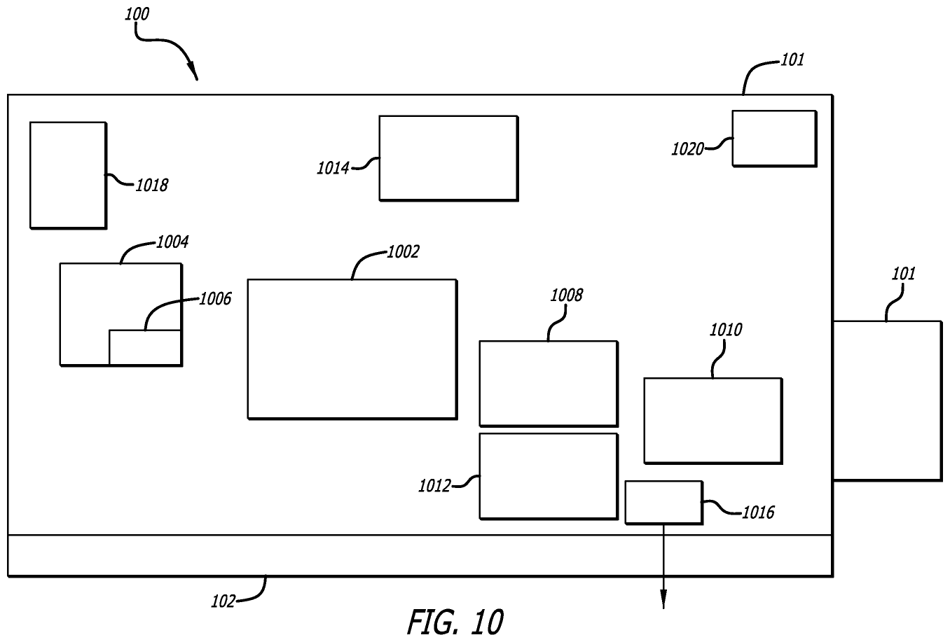

Turning now to FIG. 10, FIG. 10 is a block diagram showing the various components of the ventilator system of the present invention. The ventilator system 100 may include the following key subsystems and components: (1) a ventilator console 101; (2) UI 102 for the operator, for entry and display of information; (3) gas blender 1002 to blend oxygen and air at a selected ratio; (4) electronics system/CPU or processor 1004 to measure and control the pneumatic system and interact with the User Interface (UI); (5) software/firmware 1006 designed to facilitate, control and monitor all aspects of the product operation; (6) pneumatic system 1008 consisting of electronically driven valves to deliver respiratory ventilation to the patient; (7) sensors 1010 to monitor and control pneumatic system to provide respiratory control to patient under pressure or flow control; (8) pressure transducers or airway sensors 1012 to monitor and provide feedback, and detect excessive pressure in the patient airway path; (9) pressure switches or inlet sensors 1014 to monitor the oxygen gas and air inputs; (10) connection interface to connect to standard respiratory tube sets 1016; (11) backup battery power source 1018; (12) acoustic alert device or speakers 1020; and (13) exhalation valve assembly 136 with disposable valve component for the patient airway path that controls the flow of breathing gas from the ventilator to the patient, and from patient to atmosphere.

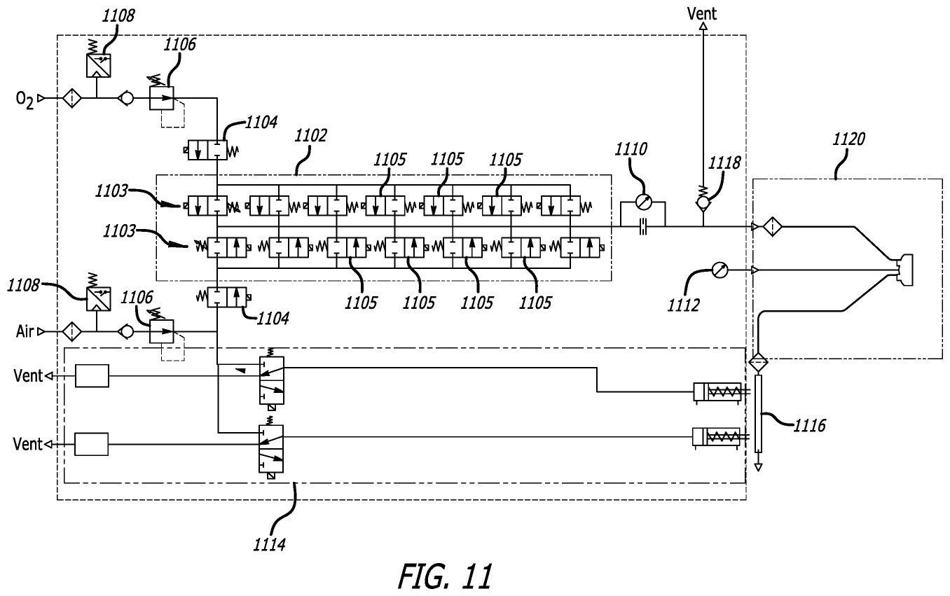

FIG. 11 is a schematic 1100 of example ventilator controls for the delivery of the mixed gas to the patient. As illustrated in FIG. 11, and as will be explained further below, the ventilator controls comprising the following: (1) air and oxygen mixing valves 1102; (2) air and oxygen isolation valves 1104; (3) pressure regulators 1106; (4) inlet filtration and sensing 1108; (5) a flowmeter 1110; (6) a patient airway sensor 1112; (7) exhalation valve actuator control pneumatics 1114; (8) exhalation valve 1116; (9) pressure relief valve 1118; and (10) breathing circuit 1120.

In this example, the ventilator architecture may provide flow control up to 101 LPM, or a total air flow. In this example, the air and oxygen mixing valves 1102 consist of a plurality of banks, each having a plurality of valves. In this example, at least two banks 1103 of solenoid valves 1105 for air and oxygen delivery are provided. Each valve bank 1103 for air and oxygen consists of at least seven (7) solenoid valves 1105 which will each provide controlled flow from 0-37 LPM per valve. Each valve 1105 in the bank has a specific orifice size to provide necessary flow with an upstream regulated pressure of 10 PSIg and total maximum flow control up to 101 LPM. In certain examples, each valve may have a different orifice size, such that each valve provides different flow capabilities, the combination of which will provide the total air flow. In some examples, at least two valves in the bank will have different orifice size, such that as many as all the solenoids in a bank each have a different orifice size, thereby allowing the operation of the different solenoids to product different flow rates as the pressure, flow, and volume of the delivered gas is required to change as the condition of the patient changes. In some examples, less than seven (7) solenoid valves 1105 may be used in one or both banks 1103, for example, 3-6 or 7 or more valves 1105 may be used in a single bank with the orifices size of the valves varying among at least two but up to all of the valves of in a bank 1103.

To provide the proper oxygen concentration, the two valve banks 1103 will control their respective flows to provide the required flow for proper mixing. These two flows combined will produce the total flow of the system which will me modulated to control system pressure.

Air and oxygen isolation valves 1104, which in this example are each solenoid, are positioned upstream of both valve banks to ensure the system can safely shutoff flow in the event one of the solenoid valves in the air and O2 mixing array fails to properly close. This will ensure safe operation of the ventilator.

Pressure regulators 1108 are also provided to regulate the high-pressure gas delivered from the hospital supply with the downstream pressure regulators. These regulators will maintain downstream pressure of 10 PSIg.

Inlet filtration and sensing 1108 is provided to filtered and monitored incoming gas prior to delivery to the regulators. The gas first enters the inline filter to remove particulates that can disrupt operation of the regulators of solenoid valves. The incoming pressure is monitored with a pressure switch to verify the pressure delivered is suitable to sustain proper ventilation. If pressure is too low, an alarm will be triggered. An inline check valve is also included to mitigate ambient gas and contaminants from entering the ventilator when the gas supply lines are disconnected.

Flow meter or flow sensor 1110 is also provided downstream of the mixing valve array 1102 to measure flow delivered to the patient, which is used for calculation of the delivered tidal volume. This flow sensor 1110 is an orifice restrictor. Differential pressure is measured across the orifice, calculating the delivered flowrate.

The patient airway sensor 1112 measures the patient airway pressure using a pressure sensor, where one port senses the pressure at the patient fitting and the other port references ambient pressure. This will provide an accurate airway pressure reading at all elevations.

An exhalation valve actuator 1116 with two pneumatic actuators is also provided to pinch the exhalation valve tubing 130. One actuator provides total closure of the exhalation valve and the second actuator provides a partial closing of the valve. The partial closure of the valve is provided to generate a higher system resistance so the flowrate maintained during PEEP pressures can be maintained at a lower range to conserve gas usage. The valve actuation is controlled by regulated gas from the air supply line with actuates a single acting piston actuator. The actuator is spring returned open to failsafe in the open position in the event of power loss or alarms triggers.

The schematic diagram also illustrates the exhalation valve 1116, which is also illustrated as part 130 in the prior illustrations. The exhalation valve 1116 is a soft silicone tubing that allows for the pneumatic actuators of the exhalation valve actuator 1116 to easily pinch the tubing closed or to a partially open position during various positions of the pressure waveform. The exhalation valve is a disposable item along with the breathing circuit.

A positive pressure relief valve 1118 is also provided as a safety device that allows for quick depressurization of the patient airway in the event airway pressure exceeds 80 cmH₂O. This valve is a passive mechanical device which is set to the desired pressure limit. This valve vents all excess gas out of the ventilator housing.

A breathing circuit for the patient is also provided for the patient, with may be an off-the shelf breathing circuit. The breathing circuit is a disposable tube set provided by the hosting hospital and is only a single use device per patient. Generally, the breathing circuit is an adult breathing size with 22 mm ISO connections.

To operate to ventilate a patient, the following additional materials are required: breath circuit with pressure line, bacterial/viral filter (attached to the expiratory limb of the patient breathing circuit), humidifier or HME or HME filter (attached to the inspiratory limb of the patient breathing circuit); oxygen (O2) monitoring device (attached to the patient breathing circuit); carbon dioxide (CO2) monitoring device (attached to the patient breathing circuit).

It will be understood, and is appreciated by persons skilled in the art, that one or more processes, sub-processes, or process steps described above may be performed by hardware and/or software. If the process is performed by software, the software may reside in software memory (not shown) in a suitable electronic processing component or system. The software in software memory may include an ordered listing of executable instructions for implementing logical functions (that is, “logic” that may be implemented either in digital form such as digital circuitry or source code or in analog form such as analog circuitry or an analog source such an analog electrical, sound or video signal), and may selectively be embodied in any computer-readable medium for use by or in connection with an instruction execution system, apparatus, or device, such as a computer-based system, processor-containing system, or other system that may selectively fetch the instructions from the instruction execution system, apparatus, or device and execute the instructions. In the context of this disclosure, a “computer readable medium” is any means that may contain, store or communicate the program for use by or in connection with the instruction execution system, apparatus, or device. The computer readable medium may selectively be, for example, but is not limited to, an electronic, magnetic, optical, electromagnetic, infrared, or semiconductor system, apparatus or device. More specific examples, but nonetheless a non-exhaustive list, of computer-readable media would include the following: a portable computer diskette (magnetic), a RAM (electronic), a read-only memory “ROM” (electronic), an erasable programmable read-only memory (EPROM or Flash memory) (electronic) and a portable compact disc read-only memory “CDROM” (optical). Note that the computer-readable medium may even be paper or another suitable medium upon which the program is printed, as the program can be electronically captured, via for instance optical scanning of the paper or other medium, then compiled, interpreted or otherwise processed in a suitable manner if necessary, and then stored in a computer memory.

It will be understood that the term “in signal communication” as used herein means that two or more systems, devices, components, modules, or sub-modules are capable of communicating with each other via signals that travel over some type of signal path. The signals may be communication, power, data, or energy signals, which may communicate information, power, or energy from a first system, device, component, module, or sub-module to a second system, device, component, module, or sub-module along a signal path between the first and second system, device, component, module, or sub-module. The signal paths may include physical, electrical, magnetic, electromagnetic, electrochemical, optical, wired, or wireless connections. The signal paths may also include additional systems, devices, components, modules, or sub-modules between the first and second system, device, component, module, or sub-module.

More generally, terms such as “communicate” and “in . . . communication with” (for example, a first component “communicates with” or “is in communication with” a second component) are used herein to indicate a structural, functional, mechanical, electrical, signal, optical, magnetic, electromagnetic, ionic or fluidic relationship between two or more components or elements. As such, the fact that one component is said to communicate with a second component is not intended to exclude the possibility that additional components may be present between, and/or operatively associated or engaged with, the first and second components

It will be understood that various aspects or details of the invention may be changed without departing from the scope of the invention. Furthermore, the foregoing description is for the purpose of illustration only, and not for the purpose of limitation—the invention being defined by the claims.

The foregoing description of an implementation has been presented for purposes of illustration and description. It is not exhaustive and does not limit the claimed inventions to the precise form disclosed. Modifications and variations are possible in light of the above description or may be acquired from practicing the invention. The claims and their equivalents define the scope of the invention.

What is claimed is:

1. A ventilator that is able to dynamical adjustment the pressure, flow, and volume of the delivered gas to a patient as the condition of the patient changes, the ventilator comprising:
a ventilator console; and
an exhalation valve housing and exhalation valve base for coupling with the exhalation valve housing to retain a flexible exhalation tube; and
at least two pistons extending through the exhalation valve base for imparting pressure on the flexible exhalation tube when actuated to restrict the flow of air through the flexible exhalation tube.

2. The ventilator of claim 1 where the at least two pistons each move an anvil to impart pressure on the walls of the flexible tube to push the walls of the flexible exhalation tube toward one another and at least partially close the flow of air through the flexible exhalation tube.

3. The ventilator of claim 1 where the at least two pistons each move an anvil to impart pressure on the walls of the flexible tube to push the walls of the flexible exhalation tube toward one another and to completely close the flow of air through the flexible exhalation tube.

4. A ventilator that is able to dynamical adjustment the pressure, flow, and volume of the delivered gas to a patient as the condition of the patient changes, the ventilator comprising:
a ventilator console;
an exhalation valve assembly; and
flow control valves, where the flow control and gas mixing valves includes at least two banks of valves, each bank has a plurality of valves where each valve has a specific orifice size that is different from at least one other valve in the respective bank of valves.

5. The ventilator of claim 4 where one of the at least of banks of valves delivers oxygen.

6. The ventilator of claim 4 where one of the at least of banks of valves delivers air.

7. The ventilator of claim 4 where each of the plurality of valves in at least one of the at least two banks of valves had a different orifice size.

8. The ventilator of claim 4 where the plurality of valves are solenoid valves.

9. The ventilator of claim 4 where the plurality of valves provide a total flow control of at least 101 LPM.

10. The ventilator of claim 4 where each bank of valves has at least seven (7) solenoid valves that each provide controlled flow from 0-37 LPM per valve, such that each valve in the bank has a specific orifice size to provide necessary flow with an upstream regulated pressure of at least 10 PSIg and a total flow control of at least 101 LPM.
